(19) United States
(12) Patent Application Publication
(10) Pub. No.: US 2016/0083453 A1
(43) Pub. Date: Mar. 24, 2016

(54) SEPARATION OF RECOMBINANT POLYCLONAL ANTIBODY MULTIMERS WITH MINIMAL SEPARATION OF MONOMERS

(71) Applicant: MEDIMMUNE, LLC,, Gaithersburg, MD (US)

(72) Inventors: Alan HUNTER, Damascus, MD (US); Timothy PABST, Damascus, MD (US); Jihong WANG, Rockville, MD (US); Xiangyang WANG, Gaithersburg, MD (US); Hongji LIU, Poolsville, MD (US)

(73) Assignee: Medlmmune, LLC, Gaithersburg, MD (US)

(21) Appl. No.: 14/890,791

(22) PCT Filed: May 12, 2014
(86) PCT No.: PCT/US14/37684

§ 371 Date: Nov. 12, 2015

Related U.S. Application Data
(60) Provisional application No. 61/822,887, filed on May 13, 2013.

(51) Int. Cl. C07K 16/00; C07K 1/16

(52) U.S. Cl. CPC C07K 16/00; C07K 1/16

(57) ABSTRACT

The invention provides a method for removing multimers from a preparation of recombinant polyclonal antibodies (rpAbs) while maintaining the ratio of monomers within a narrow range. The invention provides a method of separating recombinant polyclonal antibody multimers with minimal separation of monomers comprising subjecting a mixture comprising a plurality of monoclonal antibodies to at least one separation process selected from the group consisting of multi-modal chromatography, apatite chromatography, and hydrophobic interaction chromatography thereby producing an antibody monomer preparation that is substantially free of multimers.

### FIELD OF THE INVENTION

The invention relates to the separation of antibody multimers (multimers) from a preparation of recombinant polyclonal antibodies (rpAbs).

### BACKGROUND OF THE INVENTION

The control of multimers and multimers in recombinant biopharmaceutical preparations is of interest as these species potentially pose safety and immunogenicity concerns (Rosenberg, A. S. (2006) AAPSJ 8:59; G. Shankar, G. et al. (2007) Nat. Biotechnol. 25:555; Cordoba-Rodriguez, R. (2008) Biopharm. Int. 21:44). For recombinant monoclonal antibodies (mAbs), separation of multimers is frequently achieved using ion exchange chromatography, where monomer purity of the final antibody preparation often exceeds 99% (Suda, E. J. et al. (2009) J. Chrom. A. 1216:5256; Zhou, J. X. et al. (2007) J. Chrom. A. 1175:69; Yigzaw, Y. et al. (2009) Curr. Pharm. Biotechnol. 10:421). For polyclonal IgG preparations derived from human plasma, the level of IgG-multimers is higher, ranging from 5-18% in one study of IVIG preparations (Knezevic-Maramica, I. et al. (2003) Transfusion 43:1460). The higher level of multimers seen in commercial polyclonal WIG preparations compared to mAbs is due in part to the diverse nature of the material (e.g., range of isoelectric points and IgG subclasses). While maintaining the full diversity of plasma derived WIG is important for therapeutic reasons, it also makes it extremely difficult to separate multimers without simultaneously separating IgG monomers that differ based on characteristics such as charge (Forcer, N. et al. (2008) J. Chrom. A. 1214:59).

Recombinant polyclonal antibodies (rpAbs) represent a novel class of biopharmaceuticals that enable targeting of multiple antigens. To reduce cost, it is anticipated that rpAbs for therapeutic use will be manufactured in a single batch, where the individual component mAbs are co-expressed in the same bioreactor and purified together (Rasmussen, S. K. et al. (2012) Arch. Biochem. Biophys. 526:139).

Similar to mAbs, for rpAbs it is desirable to control multimeric species at low levels. Dissimilar to mAbs, rpAbs purification adds an additional constraint that the relative ratios of the individual component mAbs be controlled within a narrow range (T. P Frandsen, T. P. et al. (2011) Biotech. Bioeng. 108:2171). This problem represents a significant challenge as it entails separation of an undesired species (multimer) without simultaneous separation of a diverse group of mAbs representing the rpAb mixture, thus ensuring antibody relative ratios are maintained within a narrow range. Stating the problem another way, the component mAbs of the polyclonal mixture must co-purify together while the multimeric species must not. For such challenging separations, traditional approaches used for mAbs such as ion exchange chromatography may not be appropriate.

We have surprisingly discovered methods to achieve separation of recombinant polyclonal antibody multimers with minimal simultaneous separation of monomers.

### SUMMARY OF THE INVENTION

The invention provides a method of separating recombinant polyclonal antibody multimers with minimal separation of monomers comprising subjecting a mixture comprising a plurality of monoclonal antibodies to at least one separation process selected from the group consisting of multi-modal chromatography, apatite chromatography, and hydrophobic interaction chromatography thereby producing an antibody monomer preparation that is substantially free of multimers.

In some embodiments, the mixture is subjected to at least two separation processes selected from the group consisting of multi-modal chromatography, apatite chromatography, and hydrophobic interaction chromatography thereby producing an antibody monomer preparation that is substantially free of multimers.

In other embodiments, the separation process is multi-modal chromatography alone. In other embodiments, the separation process is apatite chromatography alone. In other embodiments, the separation process is hydrophobic interaction chromatography alone.

In some embodiments, the separation process is multi-modal chromatography and apatite chromatography. In some embodiments, the separation process is multi-modal chromatography and hydrophobic interaction chromatography. In some embodiments, the separation process is apatite chromatography and hydrophobic interaction chromatography. In some embodiments, the mixture is subjected to multi-modal chromatography, apatite chromatography, and hydrophobic interaction chromatography thereby separating recombinant polyclonal antibody multimers with minimal separation of monomers.

In some embodiments, the antibody preparation produced by the method is at least 90% to 91% free of multimers. In other embodiments, the antibody preparation is at least 92% to 93% free of multimers. In other embodiments, the antibody preparation is at least 94% to 95% free of multimers. In other embodiments, the antibody preparation is at least 96% to 97% free of multimers. In other embodiments, the antibody preparation is at least 98% to 99% free of multimers. In other embodiments, the antibody preparation is 100% free of multimers.

In some embodiments, the amount of any antibody monomer relative to any other antibody monomer in the rpAb mixture changes by less than 40%. In other embodiments, the amount of any antibody monomer relative to any other antibody monomer in the rpAb mixture changes by less than 30%. In other embodiments, the amount of any antibody monomer relative to any other antibody monomer in the rpAb mixture changes by less than 20%. In other embodiments, the amount of any antibody monomer relative to any other antibody monomer in the rpAb mixture changes by less than 10%. In other embodiments, the amount of any antibody monomer relative to any other antibody monomer in the rpAb mixture changes by less than 5%. In other embodiments, the amount of any antibody monomer relative to any other antibody monomer in the rpAb mixture changes by 0%.

The invention also provides a method of separating recombinant polyclonal antibody multimers with minimal separation of monomers comprising contacting a mixture comprising a plurality of monoclonal antibodies to a multi-modal chromatography resin and eluting antibody monomers from said resin with at least one elution buffer comprising a buffer species and a salt between 0 and 1 M.

The invention also provides a method of separating recombinant polyclonal antibody multimers with minimal separation of monomers comprising contacting a mixture comprising a plurality of monoclonal antibodies to an apatite chromatography resin and eluting antibody monomers from said resin with at a stepwise change or linear gradient in a salt to increase conductivity from less than 1 mS/cm to greater than 90 mS/cm or any range in-between 1 mS/cm and 90 mS/cm. For example a column may be eluted with a stepwise change in salt to increase conductivity from 5 mS/cm to 20 mS/cm.

The invention also provides a method of separating recombinant polyclonal antibody multimers with minimal separation of monomers comprising contacting a mixture comprising a plurality of monoclonal antibodies to a hydrophobic interaction chromatography resin and eluting antibody monomers from said resin with at a stepwise change or linear gradient in a salt to decrease conductivity from greater than 200 mS/cm to less than 1 mS/cm or any range in-between 200 mS/cm and 1 mS/cm. For example, a column may be eluted with a stepwise change in salt to decrease conductivity from 60 mS/cm to 10 mS/cm.

### BRIEF DESCRIPTION OF THE FIGURES

FIG. 1 shows POROS 50HS chromatography of rpAb mixtures containing mAbs A, B, and C in approximate ratios of 1:1:1.

FIG. 2 shows Capto Adhere chromatography of rpAb mixtures containing mAbs A, B, and C in approximate ratios of 1:1:1.

FIG. 3 shows Capto Adhere chromatography of rpAb mixtures containing mAbs A and B in an approximate ratio of 1:1.

FIG. 4 shows hydroxyapatite chromatography of rpAb mixtures containing mAb A and mAb B in an approximate ratio of 1:1.

FIG. 5 shows butyl chromatography of rpAb mixtures containing mAbs A and B in an approximate ratio of 1:1.

### DETAILED DESCRIPTION

### Introduction

Purification of rpAbs presents a particular challenge in that various species of multimers, or multimers may be generated when a plurality of monoclonal antibodies are co-expressed in cell culture. While various techniques are known for purifying monoclonal antibodies from cell culture, it was not expected that any of these techniques could purify monomers of monoclonal antibodies within a polyclonal antibody admixture (or mixture?) having different chemical and physical properties such as isoelectric point, (pI), hydrophobicity, and size while maintaining the relative ratios of these monoclonal antibodies in a narrow range.

### DEFINITIONS

As used herein, “Antibodies” means a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one light and one heavy chain. The variable regions of each light/heavy chain pair form an antibody binding site. The term “antibodies,” as used herein, also encompasses bi-specific antibodies.

As used herein, “apatite chromatography” means a type of separation that relies on nonspecific interactions between an analyte protein and the positively charged calcium ions and negatively charged phosphate ions on the stationary phase apatite resin. This type of chromatography includes, for example, hydroxyapatite and fluoroapatite, which interact with proteins through nonspecific interactions with calcium and phosphate ions.

As used herein, “hydrophobic interaction chromatography” means a type of separation that relies on the hydrophobic portions of an analyte protein binding to the resin under high salt conditions, but which elute under conditions of low salt.

As used herein, “minimal separation of monomers” refers to the removal of only a small amount of antibody monomers from the original mixture relative to any other antibody monomer in the mixture. Generally, the amount of separation will be less than 40% of the monomers from the original mixture relative to any other monomer. Preferably, the amount will be less than 30%. More preferably, the amount will be less than 20%. More preferably, the amount will be less than 10%. More preferably still, the amount will be less than 5%. In some embodiments, there will be no separation of monomers (0%).

As used herein, “Monoclonal antibody (mAb)” refers to an antibody in a clonal preparation in which each of the antibodies in the preparation has a single specificity, binding to the identical epitope.

As used herein, “Monomer” means a single antibody molecule without multimer.

As used herein, “Multimer” means high molecular weight aggregates of antibodies.

As used herein, “Multi-modal chromatography” refers to a technique that relies on more than one mode of interaction between the stationary phase and analytes to effect a separation. For example, multimodal chromatography may rely on one or more of the following types of chromatography in combination with another of these interactions: ion exchange chromatography (IEC), hydrophobic interaction chromatography (HIC), reversed phase liquid chromatography (RPLC), and size exclusion chromatography (SEC).

As used herein, “Recombinant Polyclonal Antibodies (rpAbs)” means a plurality of monoclonal antibodies in admixture. In the methods of the present invention, the individual component mAbs are co-expressed in the same bioreactor and purified together or expressed in separate bioreactors and mixed together at any point during the purification process.

As used herein, “stepwise change” as it relates to elution conditions means an instantaneous or very rapid change in conductivity, typically occurring in less than 1 column volume, to elute an rpAb mixture from a resin.

As used herein, “linear gradient” as it relates to elution conditions means a gradual change in conductivity occurring over a fixed duration, typically between 1 and 50 column volumes.

As used herein, “buffer species” refers to a weak acid and its conjugate base or a weak base and its conjugate acid that can resist pH changes. Buffer species may be selected from a list including but not limited to acetate, phosphate, citrate, tris, and bis-tris.

As used herein “salt” is a combination of an anion and a cation. Cations may be selected from a list including but not limited to sodium, ammonium, calcium, magnesium, and potassium. Anions may be selected from a list including but not limited to chloride, phosphate, citrate, acetate, and sulfate.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

(A) Separation of rpAbs

Recombinant polyclonal antibodies (rpAbs) comprising a diversity of monoclonal antibodies, each with their attendant chemical properties, present a significant challenge for purification. Surprisingly, it has been found that some separation techniques (specifically, hydrophobic interaction chromatography, multi-modal chromatography and apatite chromatography), either alone or in combination, can separate monomers from the mixtures containing species of multimers of these antibodies, while maintaining the ratio of individual monoclonal antibodies in a narrow range at high purity of monomers.

In general, the mixture of rpAbs would first be subjected to one or more chromatographic separation techniques to remove process related impurities prior to removal of multimers. The choice of chromatographic techniques common in the art may include Protein A affinity chromatography, to capture the rpAb mixture from the clarified cell culture media, and anion exchange, to remove additional process-related species. These initial purification steps do not change the ratio of individual mAb components, and they do not significantly reduce the level of multimers in the rpAb mixture.

### (B) Separation Techniques

1. Multi-Modal Chromatography

Multi-modal chromatography may be carried out using commercially available resins (such as that sold by GE Healthcare Life Sciences under the name “Capto Adhere”) and by any multi-modal buffer system known in the art. In the method of the present invention, multi-modal chromatography utilizes resins that incorporate ion exchange and hydrophobic interaction groups. The resin used may be packed into a column, prepared as a fluidized bed column or as a batch preparation. Multi-modal chromatography may be operated under bind and elute conditions, where monomers and multimers are both bound to the column and then monomers are selectively eluted with a change in salt concentration and/or pH, or under flowthrough conditions, where the multimers are bound to the column while the individual monomers largely remain in the column flowthrough. A person of ordinary skill in the art will be able to choose conditions for both options.

As a non-limiting example operated under flowthrough conditions, an equilibration buffer may be composed of 25 mM acetate, 100 mM sodium chloride, pH 5.0. In some embodiments, the buffer comprises 5 to 200 mM acetate. In some embodiments, the buffer comprises 10 to 100 mM acetate. In some embodiments, the buffer comprises 15 to 35 mM acetate. In some embodiments, the buffer comprises 25 mM acetate. In some embodiments, the buffer comprises 0 to 1 M salt. In some embodiments, the buffer comprises 0 to 1 M sodium chloride. In some embodiments, the buffer comprises 50 to 500 mM sodium chloride. In some embodiments, the buffer comprises 80 to 120 mM sodium chloride. In some embodiments, the buffer comprises 90 to 110 mM sodium chloride. In some embodiments, the buffer comprises 100 mM sodium chloride. In some embodiments, the pH is in the range of about 3.0 to 6.0. In some embodiments, the pH is in the range of about 4.5 to 5.5. In some embodiments, the pH is 5.0.

In another non-limiting example operated under flowthrough conditions, the equilibration buffer that may be used is composed of 50 mM tris, 100 mM sodium chloride, pH=7.25. In some embodiments, the buffer comprises 5 to 200 mM tris. In some embodiments, the buffer comprises 10 to 100 mM tris. In some embodiments, the buffer comprises 40 to 60 mM tris. In some embodiments, the buffer comprises 50 mM tris. In some embodiments, the buffer comprises 0 to 1 M salt. In some embodiments, the buffer comprises 0 to 1 M sodium chloride. In some embodiments, the buffer comprises 50 to 500 mM sodium chloride. In some embodiments, the buffer comprises 80 to 120 mM sodium chloride. In some embodiments, the buffer comprises 90 to 110 mM sodium chloride. In some embodiments, the buffer comprises 100 mM sodium chloride. In some embodiments, the pH is in the range of about 6.0 to 10.0. In some embodiments, the pH is in the range of about 7.0 to 9.0. In some embodiments, the pH is 7.1 to 7.5. In some embodiments, the pH is 7.25.

The loading buffer is substantially the same as the equilibration buffer (with the rpAbs)

The resin may be washed in a buffer that is substantially the same as the loading buffer (without the rpAbs).

The protein in the column flowthrough may be collected based on absorbance at 25 mAU on the leading and tailing side of the product peak.

2. Apatite Chromatography

Apatite chromatography may be conducted using various buffers for loading, washing and elution. The resin used may be packed into a column, prepared as a fluidized bed column or as a batch preparation. Apatite chromatography may be operated under bind and elute conditions, where monomers and multimers are both bound to the column and then monomers are selectively eluted with a change in salt concentration and/or pH, or under flowthrough conditions, where the multimers are bound to the column while the individual monomers largely remain in the column flowthrough. A person of ordinary skill in the art will be able to choose conditions for both options.

As a non-limiting example under bind and elute conditions, the equilibration buffer that may be used is composed of 10 mM phosphate, 100 mM NaCl, pH 7.0. In some embodiments, the buffer comprises about 1 to 100 mM sodium phosphate. In some embodiments, the buffer comprises 2 to 50 mM phosphate. In some embodiments, the buffer comprises about 5 to 15 mM phosphate. In some embodiments, the buffer comprises 10 mM phosphate. In some embodiments, the buffer comprises about 0 to 100 mM salt. In some embodiments, the buffer comprises about 0 to 100 mM sodium chloride. In some embodiments, the buffer comprises 1 to 50 mM sodium chloride. In some embodiments, the buffer comprises 5 to 15 mM sodium chloride. In some embodiments, the buffer comprises 10 mM sodium chloride. In some embodiments, the pH is in the range of about 6.2 to 8.0. In some embodiments, the pH is in the range of about 6.8 to 7.2. In some embodiments, the pH is 7.0.

The loading buffer is substantially the same as the equilibration buffer (with rpAbs)

The resin may be washed in a buffer that is substantially the same as the loading buffer (without the rpAbs).

For elution, the buffer may be a higher ionic strength (higher than the equilibration and loading buffer) phosphate buffer comprising about 0.05 to 3 M NaCl having a pH in the range of about 6.2 to 8.0. In some embodiments, the buffer comprises about 1 to 100 mM phosphate. In some embodiments, the buffer comprises 2 to 50 mM phosphate. In some embodiments, the buffer comprises about 5 to 15 mM phosphate. In some embodiments, the buffer comprises 10 mM phosphate. In some embodiments, a step wise or linear gradient of salt is used to elute in which the step or gradient is from about 0 M to 3 M salt. In some embodiments, a step wise or linear gradient of sodium chloride is used to elute in which the step or gradient is from about 0 M to 3 M sodium chloride. In some embodiments, a step wise or linear gradient of sodium chloride is used to elute in which the step or gradient is from about 1 mM to 1 M sodium chloride. In some embodiments, the pH is in the range of about 6.5 to 7.5. In some embodiments, the pH is in the range of about 6.8 to 7.2. In some embodiments, the pH is 7.0.

3. Hydrophobic Interaction Chromatography

Hydrophobic interaction chromatography may be conducted using various buffers for loading, washing and elution. The resin used may be packed into a column, prepared as a fluidized bed column or as a batch preparation. Hydrophobic interaction chromatography may be operated under bind and elute conditions, where monomers and multimers are both bound to the column and then monomers are selectively eluted with a change in salt concentration and/or pH, or under flowthrough conditions, where the multimers are bound to the column while the individual monomers largely remain in the column flowthrough. A person of ordinary skill in the art will be able to choose conditions for both options.

As a non-limiting example under bind and elute conditions, the equilibration buffer that may be used is composed of a phosphate buffer comprising 0.6 M sodium sulfate and a pH of 7.0. In some embodiments, the buffer comprises about 5 to 200 mM phosphate. In some embodiments, the buffer comprises about 10 to 100 mM phosphate. In some embodiments, the buffer comprises about 15 to 25 mM phosphate. In some embodiments, the buffer comprises 20 mM phosphate. In some embodiments, the buffer comprises about 0.2 to 2 M salt. In some embodiments, the buffer comprises about 0.3 to 1 M salt. In some embodiments, the buffer comprises about 0.5 to 0.7 M salt. In some embodiments, the buffer comprises 0.5 to 0.7 M sodium sulfate. In some embodiments, the buffer comprises 0.6 M sodium sulfate. In some embodiments, the pH is in the range of about 6.2 to 8.0. In some embodiments, the pH is in the range of about 6.8 to 7.2. In some embodiments, the pH is 7.0

The loading buffer is substantially the same as the equilibration buffer (with rpAbs)

The resin may be washed in a buffer that is substantially the same as the loading buffer (without the rpAbs).

For elution, the buffer may be a lower ionic strength phosphate buffer (i.e. lower than the equilibration and loading buffer) comprising about 0 to 0.6 mM sodium sulfate and a pH of about 7.0. In some embodiments, the buffer comprises 0.1 to 0.5 mM salt. In some embodiments, a step wise or linear gradient of decreasing salt is used to elute in which the stepwise or gradient is from about 1 M to 0 M salt. In some embodiments, a step wise or linear gradient of decreasing sodium sulfate is used to elute in which the step or gradient is from about 0.8 M to 0 M salt. In some embodiments, a step wise or linear gradient of decreasing sodium sulfate is used to elute in which the step or gradient is from about 0.6 M to 0 M salt. In some embodiments, a step wise or linear gradient of decreasing sodium sulfate is used to elute in which the step or gradient is from about 0.6 M to 0 M sodium sulfate. In some embodiments, the pH is in the range of about 6.2 to 8.0. In some embodiments, the pH is in the range of about 6.8 to 7.2. In some embodiments, the pH is 7.0.

The product may be collected based on absorbance of 25 mAU on the leading side of the peak and 25 mAU on the tailing side of the peak.

In the method of the invention, the multimers are removed such that the antibody preparation is at least 90% free of multimers. In some embodiments, the antibody preparation is at least 91% free of multimers. In some embodiments, the antibody preparation is at least 92% free of multimers. In some embodiments, the antibody preparation is at least 93% free of multimers. In some embodiments, the antibody preparation is at least 94% free of multimers. In some embodiments, the antibody preparation is at least 95% free of multimers. In some embodiments, the antibody preparation is at least 96% free of multimers. In some embodiments, the antibody preparation is at least 97% free of multimers. In some embodiments, the antibody preparation is at least 98% free of multimers. In some embodiments, the antibody preparation is at least 99% free of multimers. In some embodiments, the antibody preparation is 100% free of multimers.

Resins that may be used in the methods of the invention are well known in the art and are commercially available.

The method of separating recombinant polyclonal antibody multimers may employ a multi-modal chromatography resin wherein the rpAbs are contacted to the resin and the multimers are bound to the resin while the monomers are collected in the column flowthrough.

The method of separating recombinant polyclonal antibody multimers may employ a multi-modal chromatography resin wherein the rpAbs are bound to the resin and the monomers eluted from the resin using at least one elution buffer, wherein the elution buffer is comprising a buffer species and a salt between 0 and 1 M

The method of separating recombinant polyclonal antibody multimers may employ a multi-modal chromatography resin wherein the rpAbs are contacted to the resin and the multimers are bound to the resin while the monomers are collected in the column flowthrough.

The method of separating recombinant polyclonal antibody multimers may employ an apatite chromatography resin wherein the rpAbs are bound to the resin and the monomers eluted from the resin using at least one elution buffer, wherein the elution buffer is a stepwise change or linear gradient in a salt to increase conductivity from less than 1 mS/cm to greater than 90 mS/cm or any range in-between 1 mS/cm and 90 mS/cm.

The method of separating recombinant polyclonal antibody multimers may employ a hydrophobic interaction chromatography resin wherein the rpAbs are bound to the resin and the monomers eluted from the resin using at least one elution buffer, wherein the elution buffer is a stepwise change or linear gradient in a salt to decrease conductivity from greater than 200 mS/cm to less than 1 mS/cm or any range in-between 200 mS/cm and 1 mS/cm.

The method may also comprise a combination of these three separation techniques under these specific conditions.

Generally one would consider the pI of the antibodies and hydrophobicity profile to guide bind and elute conditions and flowthrough conditions as will be known to those of skill in the art.

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function.

### EXAMPLES

### A. Materials and Methods

1. Chemicals

All chemicals are USP grade or equivalent.

2. mAbs and rpAb Mixtures

Monoclonal antibodies were expressed and purified using cell culture and purification techniques commonly employed in biotechnology. Following standard cell culture procedures using widely available cell lines such as CHO or NS0, purification of each mAb included at least Protein A capture and an ion exchange column to remove process related impurities. The individual mAb properties are summarized in Table 1 below. To generate rpAb mixtures, the individual mAbs were then mixed in approximate ratios of 1:1 or 1:1:1 (by mass), for two and three mAb rpAb mixtures, respectively. To obtain the desired level of multimers of individual mAbs in the rpAb mixtures, purified mAbs containing high or low multimer levels were first combined in appropriate ratios to give the correct multimer level prior to combining individual mAbs. This resulted in an rpAb mixture with well-defined composition of mAb ratios and multimer levels.

TABLE 1
Summary of mAb properties

| mAb | pI^{a} | Extinction Coefficient (mg/mL)⁻¹cm⁻¹ |
|---|---|---|
| A | 9.4 | 1.47 |
| B | 9.4-9.5 | 1.44 |
| C | 7.1 | 1.61 |
| D | 7.1-7.3 | 1.40 |

3. rpAb Total Protein Concentration Measurements

Protein concentrations of rpAb mixtures were measured by absorbance at 280 nm using a Nanodrop 2000c from Thermo (Wilmington, Del.). For each mixture, the extinction coefficient was estimated using a weighted average of the individual mAb components (1:1 or 1:1:1 mixtures). Extinction coefficients of individual mAbs can be found in Table 1.

4. Cation Exchange Chromatography

Cation exchange chromatography (CEX) using POROS HS50 (Life Technologies, Location) was carried out under typical bind and elute conditions in small scale chromatography columns with 20 cm bench heights. All runs were conducted using an AKTA Explorer liquid chromatography system from GE Healthcare (Piscataway, N.J. USA) and the column was operated at 300 cm/h. The column was equilibrated with 25 mM acetate, 25 mM sodium chloride, pH 5.0 and then loaded to 30 g of protein/L of resin using the total protein concentration. After loading, the column was re-equilibrated and then eluted in a linear gradient of sodium chloride from 25 mM to 260 mM over 20 column volumes. The product peak was collected based on absorbance criteria of 25 mAU on the leading and tailing side of the product peak.

5. Multi-Modal Chromatography

Multi-modal chromatography (MMC) using Capto Adhere (GE Healthcare, Piscataway, N.J. USA) was carried out under typical flow through conditions in small chromatography columns packed to 20 cm bed height. All runs were conducted using an AKTA Explorer liquid chromatography system from GE Healthcare (Piscataway, N.J. USA) and the column was operated at 300 cm/h. The column was equilibrated with 25 mM acetate, 100 mM sodium chloride, pH 5.0 (for mixtures of mAb A, B, and C) or with 50 mM tris, 100 mM sodium chloride, pH 7.25 (mAb A and B mixtures). The column was loaded to 50 g of protein/L of resin using the total protein concentration and the re-equilibrated with the equilibration buffer. The product peak was collected based on absorbance criteria of 25 mAU on the leading and tailing side of the product peak.

6. Hydrophobic Interaction Chromatography

Hydrophobic interaction chromatography (HIC) using Toyopearl Butyl 650M from Tosoh Bioscience (King of Prussia, Pa. USA) was carried out under typical bind and elute conditions in small scale chromatography columns with 20 cm bench heights. All runs were conducted using an AKTA Explorer liquid chromatography system from GE Healthcare (Piscataway, N.J. USA) and the column was operated at 300 cm/h. The column was equilibrated with 25 mM phosphate, 0.6 M sodium sulfate, pH 7.4. Load was prepared by diluting 1 part (by volume) protein solution with 1 part 25 mM phosphate, 1.2 M sodium sulfate, pH 7.4 and then the column was loaded to 10 g of protein/L of resin using the total protein concentration (described above). After loading, the column was re-equilibrated with equilibration buffer and then eluted in a linear gradient of sodium sulfate from 0.6 M to 0 mM sodium sulfate over 20 column volumes. The product peak was collected based on absorbance criteria of 25 mAU on the leading side of the peak and 100 mAU on the tailing side of the product peak.

7. Hydroxyapatite Chromatography

Hydroxyapatite chromatography using Ceramic Hydroxyapatite Type I from Bio-Rad Laboratories (Hercules, Calif., USA) was carried out under typical bind and elute conditions in small scale chromatography columns with 20 cm bench heights. All runs were conducted using an AKTA Explorer liquid chromatography system from GE Healthcare (Piscataway, N.J. USA) and the column was operated at 300 cm/h. The column was equilibrated with 10 mM phosphate, pH 7.0 and then loaded to 20 g of protein/L of resin using the total protein concentration (described above). After loading, the column was re-equilibrated and then eluted in a linear gradient of sodium chloride from 0 to 1 M sodium chloride over 20 column volumes. The product peak was collected based on absorbance criteria of 25 mAU on the leading side of the peak and 50 mAU on the tailing side of the product peak.

8. Analytical Size Exclusion Chromatography (SEC-HPLC)

Analytical high performance size exclusion chromatography (SEC-HPLC) was performed using a TSK-GEL G3000SW_{XL} obtained from Tosoh Biosciences (Location) with an Agilent 1200 HPLC system (Palo Alto, Calif., USA). The mobile phase was 0.1 M sodium phosphate, 0.1 M sodium sulfate, pH 6.8 at 1 mL/min for 20 minutes. Samples of 45 ug were injected neat and the column was calibrated using molecular weight standards from Bio-Rad (Hercules, Calif. USA). The elution profile was monitored using a spectrophotometer at 280 nm and data was collected and analyzed using ChemStation software from Agilent.

9. Analytical Reversed Phase Chromatography (RP-HPLC)

Analytical RP-HPLC was performed with a PLRP-S column (8 μm particle, 4000 A, 2.0×150 mm) purchased from Michrom Bioresources, Inc. (Auburn, Calif., USA) connected to a Waters ACQUITY UPLC H-Class Bio system (Milford, Mass., USA). Three Eluents were used to generate the appropriate mobile phase and gradient tailored to each type of protein mixture. They were: water (Eluent A), acetonitrile (Eluent B) and 2% trifluoroacetic acid (TFA) in water (Eluent C). During each elution, the percentage of Eluent C was kept constant (TFA concentration: 0.02-0.1%) while the ratio of Eluents B over A was increased to form a desired gradient. The flow rate was set at 0.2 ml/min and the column temperature was maintained at 70° C. The elution of each protein mixture was monitored with a photodiode array detector and the peak responses acquired at either 280 nm or 220 nm were selected for quantitation. The concentration of each protein in samples was determined by injecting a standard solution prepared with the reference standard of the same protein.

### Example 1

### Cation Exchange Chromatography (mAb A/B/C)

Cation exchange chromatography (CEX) is often used for mAb multimer removal. Under typical bind and elute conditions, the multimeric species are more strongly retained on the column than monomeric species and require higher concentrations of salt to elute. For monomer/multimer separations, the most common technique for elution is a stepwise change or linear gradient of increasing salt that can be employed to exploit the subtle difference in binding between the various species and the resin. A less common technique is to use increasing pH to elute the monomer and then the multimers. Depending on the difficulty of the monomer/multimer separation the multimers may appear as a separate peak (complete resolution) or as a shoulder on the tailing side of the monomer peak (less resolved). In either case, the multimer can be removed from the mixture by cutting the product peak as to not include the multimers.

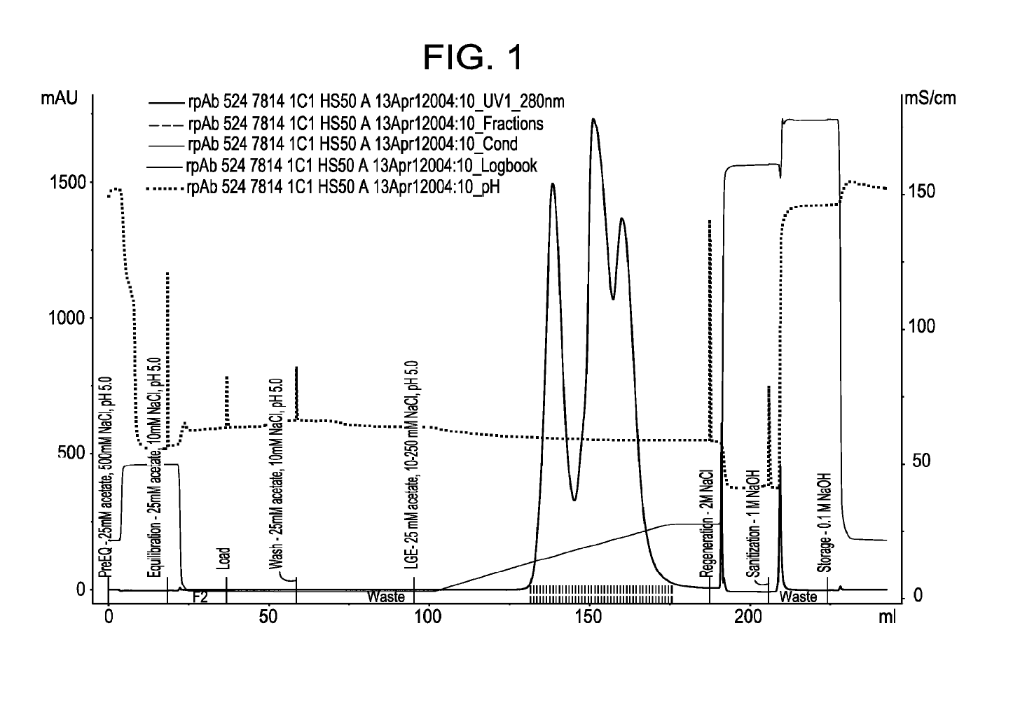

For CEX, the same techniques can be employed to remove multimers from monomers in rpAb mixtures using cation exchange chromatography. An example of rpAb purification with a mixture of mAb A, B and C, using cation exchange is shown in FIG. 1 and summarized in Table 2. For this rpAb mixture, the individual mAbs are combined in an approximate ratio of 1:1:1 and the multimers are mainly from mAb C, with a very low level of mAb B multimers and negligible levels of mAb A multimers. When the rpAb mixtures is loaded and eluted from the column 3 main peaks are observed, each peak corresponding to an individual mAb. For this rpAb mixture mAb C eluted first, followed by mAb A, and finally mAb B. The elution order was confirmed by injecting individual mAbs in place of the rpAb mixture. Separation of the multimers (mainly from mAb C) in the rpAb mixture is not easily observed in the chromatogram in FIG. 1; however, an injection of mAb C alone confirmed that the monomer eluted first, and multimers eluted later in the gradient, as expected. Under these conditions, the multimers of mAb C co-elute with the monomer of mAb A and mAb B, and thus become difficult to remove without significantly changing the mAb ratios in the rpAb mixture. It should be noted that the entire elution pool was collected (with an absorbance collection criteria of >25 mAU on the ascending and descending side of the elution peak). As can be seen in Table 2, the multimer level remains relatively unchanged from load to pool, as expected due to the co-elution of mAb C multimers with mAb A and mAb B monomers. In order to remove multimers of mAb C, one would have to also remove monomers of mAb A and/or mAb B (due to the co-elution of these species with the multimers of mAb C). These results suggest that cation exchange chromatography is not a viable option for this rpAb mixture.

TABLE 2
Summary of POROS 50HS chromatography
of rpAb mixtures of mAbs A, B, and C.

| | SEC-HPLC | Monomer Yield |
|---|---|---|
| Sample | (% Multimer) | (%) |
| Load | 4.2% | — |
| Pool | 3.7% | 100.0% |

### Example 2

### Multi-Modal Chromatography (mAb A/B/C)

Multi-modal chromatography is a unique mode of chromatography that is a hybrid of two (or more) different modes of chromatography and can be utilized in either mode, depending on how the column is operated. In the literature, the most common multi-modal chromatography resins incorporate ligands that have both ion exchange properties as well as with hydrophobic interaction properties over a wide range of pH values. Due to the unique ionic and hydrophobic properties of these ligands, multi-modal resins have been used in the separation of mAb multimers from mAb monomers. Since typical mAbs have basic isoelectric points, multi-modal resins that have CEX/HIC ligands are typically operated in bind and elute mode where the product is bound to the column at low pH and lower salt concentrations and then eluted with increased salt and/or increased pH. One example for a minibody purification showed that dimers and multimers were strongly bound and eluted in the high salt strip peak (P. Gagnon, P. et al. (2010) Bioprocess Int. 8:26). For multi-modal resins that have AEX/HIC ligands, mAbs can be processed in bind and elute mode or in flowthrough mode. When operated in flowthrough mode, the operating conditions are chosen such that the mAb monomer does not bind to the resin while the multimers bind strongly, thus removing multimers from the feed stream. Examples in the literature are common, for example Chen et al. and Eriksson et al. both describe a Capto Adhere flow-through step to remove high molecular weight species (J. Chen, J. (2010) J. Chrom. A. 1217:216; Eriksson, K. et al. (2009) Bioprocess Int. 7:52). While multimer removal using multi-modal chromatography is common in mAb purifications, applying multi-modal chromatography to remove multimers in rpAb mixtures is not as straight-forward. Due to the complex nature of the interactions between individual mAb species in a rpAb mixture and the multi-modal ligand, it is not obvious that conditions can be optimized to selectively remove multimers from monomers, while simultaneously keeping mAb ratios constant.

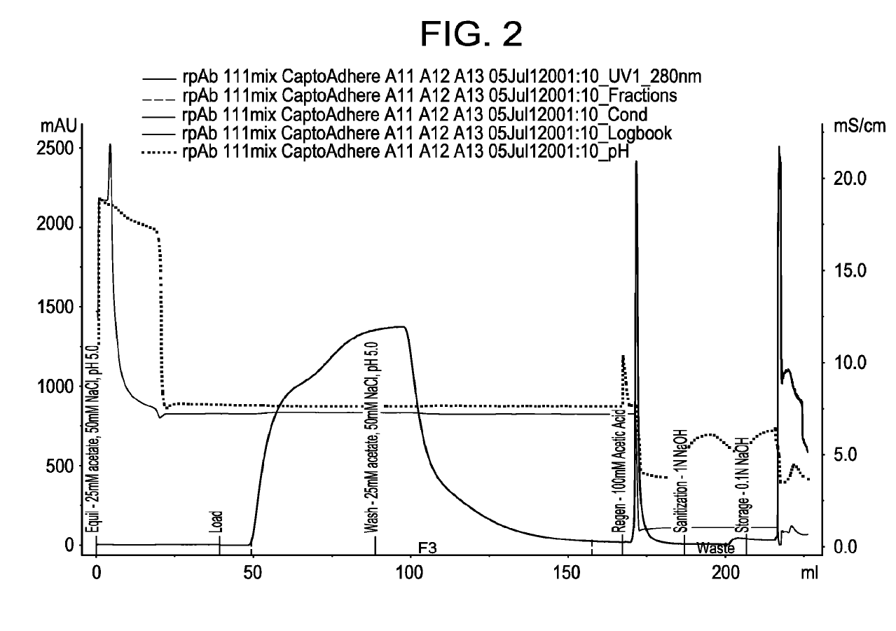

To test the ability of multi-modal chromatography to remove multimers in rpAb mixtures, we investigated purification of a mixture of mAb A, B, and C (in an approximate ratio of 1:1:1) using Capto Adhere in flowthrough mode. For this mixture, the multimers are mainly from mAb C, with very low levels of mAb B multimers and negligible levels of mAb A multimers. This mixture is nearly identical to the mixture that was used in Example 1 for CEX chromatography. FIG. 2 shows the Capto Adhere chromatogram of the rpAb mixture. Unlike bind and elute CEX, the Capto Adhere chromatogram in FIG. 2 does not show any obvious signs of separation of mAb species, which is important in rpAb purification since the mAb ratios must remain relatively constant. Table 3 summarizes the load and pool analytical data for the Capto Adhere chromatography run. As can be seen in Table 3, multimers were reduced from 3.4% in the load, to 0.8% in the Capto Adhere pool. Under these optimized load conditions (pH 5.0, 100 mM NaCl), the multimers are more strongly retained and likely appear in the low pH strip peak seen in the chromatogram. As can be seen in Table 3, the ratio of mAbs B and C to mAb A (B:A and C:A) remains very close to 1.00 before and after Capto Adhere purification. It should be noted that the ratios are based on RP-HPLC concentrations of individual mAbs, and do include contributions from both monomer and multimers. Therefore, the removal of mAb C multimers during Capto Adhere chromatography is reflected in the slight decrease in the ratio of C:A before and after Capto Adhere chromatography.

TABLE 3
Summary of Capto Adhere chromatography
of rpAb mixtures of mAb A, B, and C.

| | SEC-HPLC | mAb ratio | | Monomer Yield |
|---|---|---|---|---|
| Sample | (% Multimer) | (B:A) | (C:A) | (%) |
| Load | 3.4% | 0.95 | 1.03 | — |
| Pool | 0.8% | 0.95 | 0.90 | 100.8% |

### Example 3

### Multi-Modal Chromatography (mAb A/B)

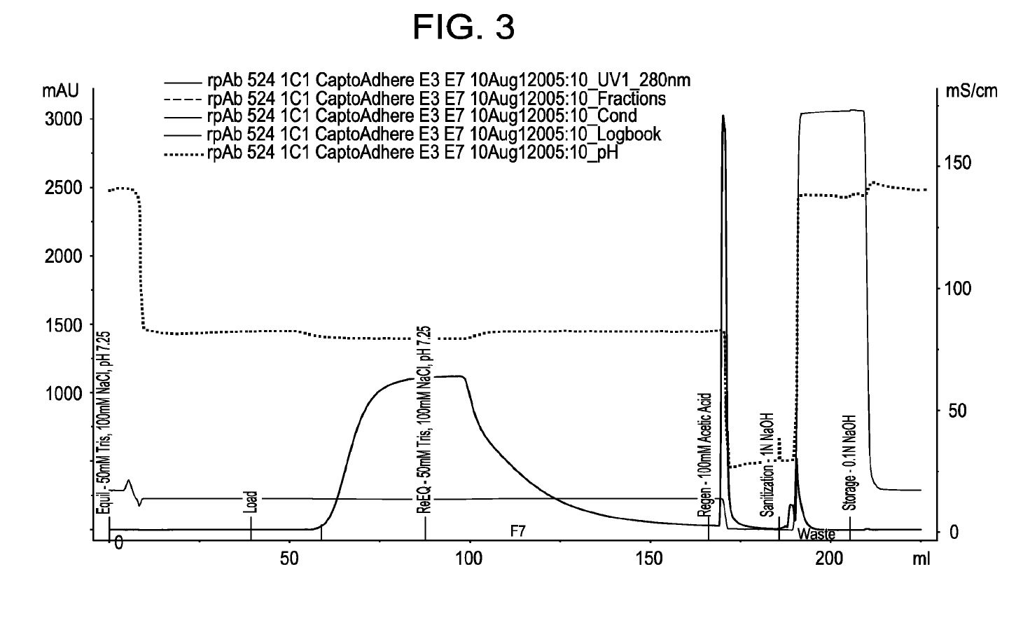

To further demonstrate the use of multi-modal chromatography for the removal of multimers from rpAb mixtures using multi-modal chromatography in flow through mode, a second rpAb mixture was investigated. FIG. 3 shows the Capto Adhere chromatogram for a 1:1 mixture of mAb A and B. As can be seen in FIG. 3, the chromatogram looks like a typical flow-through chromatogram, with no distinct separation of individual mAb species observed under the operating conditions selected (pH 7.25, 100 mM NaCl). Compared to the chromatogram in FIG. 2, the profile is very similar, with an absorbance peak in the regeneration step (0.1 M acetic acid) that represents mostly multimers.

Table 4 summarizes the load and pool samples for the Capto Adhere chromatography. In this example, the total multimer levels are higher than the previous example and the multimers in the mixture are from both mAbs, in similar levels (i.e. ˜3.5% multimers from each mAb). The combined multimer level measured in the load was 6.9%. Similar to the previous example, Capto Adhere chromatography is a very effective tool for multimer removal with this mAb mixture. As can be seen in Table 4, multimer levels were reduced from 6.9% to 0.4% by SEC-HPLC and the monomer yield is high (96.1%). This indicates that multimers from different mAbs (mAbs A or B in this case) can be removed simultaneously without compromising on monomer step yield. At the same time, the ratio of mAb B to mAb A remains relatively constant (0.99 in the load vs. 0.97 in the pool). This separation example reinforces the novelty and importance of multi-modal chromatography for removal of multimers from rpAb mixtures while keeping the individual mAb ratios constant.

TABLE 4
Summary of Capto Adhere chromatography
of rpAb mixtures of mAbs A and B.

| | SEC-HPLC | mAb ratio | Monomer Yield |
|---|---|---|---|
| Sample | (% Multimer) | (B:A) | (%) |
| Load | 6.9% | 0.99 | — |
| Pool | 0.4% | 0.97 | 96.1% |

### Example 4

### Hydroxyapatite Chromatography (mAb C/D)

Hydroxyapatite chromatography is unique chromatography media that is comprised of calcium and phosphate, which can bind proteins by cation exchange (through the phosphate ions in the resin) as well as through metal coordination (via the calcium ions in the resin). Hydroxyapatite has been widely used in the purification of protein for some time, and more recently hydroxyapatite has become a popular choice for multimer removal in mAb purification (Gagnon, P. (2009) New Biotechnol. 25:287; Gagnon, P. et al. (2009) J. Sep. Sci. 32:3857). When used in mAb purification, the column is typically equilibrated with a phosphate buffer containing low concentrations of sodium chloride at or near neutral pH. Under these conditions, the monomer and multimers typically bind to the column, with the multimer being more strongly bound. The product is eluted from the column by increasing the phosphate or NaCl concentration (NaCl tends to be more widely used elution technique) in a gradient or step fashion. If optimized, the separation of monomer and multimer can be very effective. While multimer removal using hydroxyapatite chromatography is common in mAb purifications, applying hydroxyapatite chromatography to remove multimers in rpAb mixtures is not as straight-forward. Like CEX, it is hard to predict a priori the separation of monomeric mAbs from multimeric mAbs or other mAb species based on cationic interactions alone. With the added complexity of the metal coordination interactions in hydroxyapatite, it becomes even more difficult to predict how rpAb separations will occur. Thus, it is not obvious that optimal conditions can be selected such that multimers are removed while simultaneously keeping mAb ratios constant.

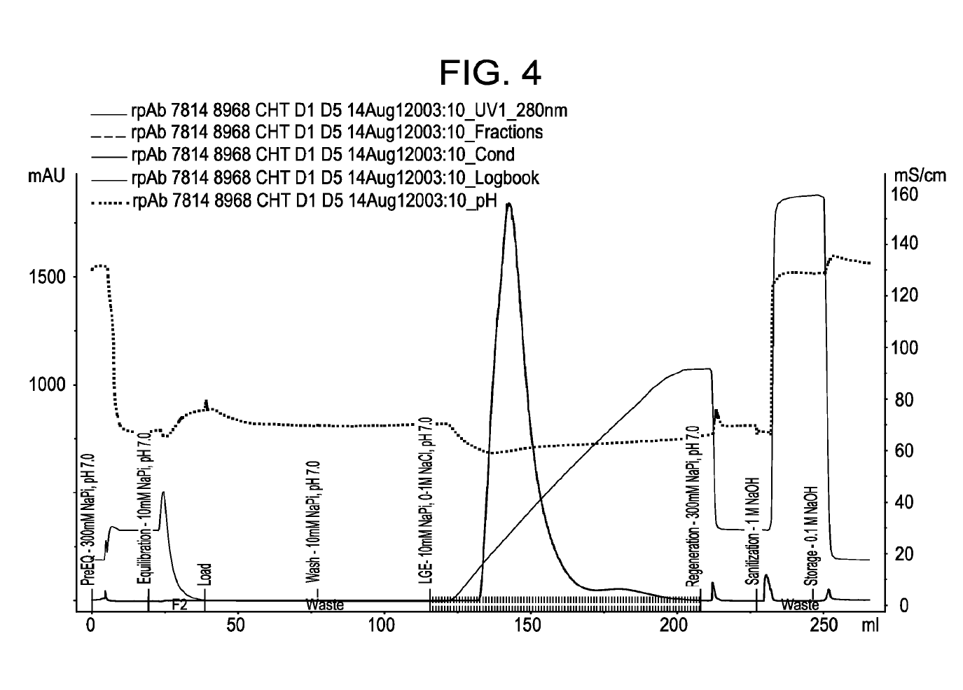

To test the ability of hydroxyapatite chromatography to remove multimers in rpAb mixtures, we investigated purification of a mixture of mAb C and D (in an approximate ratio of 1:1) using Ceramic Hydroxyapatite (Type I) in bind and elute mode with NaCl linear gradient elution. For this mixture, the multimers are mostly from mAb C, with only minor contributions of multimers from mAb D. FIG. 4 shows the Capto Adhere chromatogram of the rpAb mixture. As can be seen in the chromatogram, the mAb monomers co-elute in a single peak with no separation of the mAbs observed. If there was separation of the individual mAbs, multiple peaks with approximately similar areas would have been observed (as seen in the CEX profile in FIG. 1). Injections of the individual mAbs confirm the similar elution position within the NaCl gradient (data not shown). A small peak that elutes after the monomer peak was observed, and this peak was shown to be multimers by SEC-HPLC. Based on the chromatogram, hydroxyapatite is capable of separating multimers from monomer without separating the individual mAbs. It should also be noted that the separation was done so under conditions that still resulted in high monomer yield (96.8%). Table 5 summarizes the load and pool analytical data. As can be seen in Table 5, multimers were reduced from 4.1% in the load, to 0.4% in the hydroxyapatite pool. At the same time, the ratio of mAb D to mAb C (D:C) remained relatively constant before (0.96) and after (1.01) hydroxyapatite chromatography. As mentioned previously, there is some change in the ratio due to the removal of multimers since the ratio is determined using RP-HPLC concentrations which include both monomeric and multimeric species. Overall, hydroxyapatite has been shown to be an effective tool for multimer removal in rpAb mixtures.

TABLE 5
Summary of Hydroxyapatite chromatography
of rpAb mixtures of mAb C and D.

| | SEC-HPLC | mAb ratio | Monomer Yield |
|---|---|---|---|
| Sample | (% Multimer) | (D:C) | (%) |
| Load | 4.1% | 0.96 | — |
| Pool | 0.4% | 1.01 | 96.8% |

### Example 5

### Hydrophobic Interaction Chromatography (mAb A/B)

Hydrophobic interaction chromatography (HIC) is a common mode of chromatography that separates protein based on differences in hydrophobicities. HIC has been widely used in the purification of protein for some time, and has been documented as an option for multimer removal for mAb purification (Chen, J. et al. (2008) J. Chrom. A. 1177:272). When used for mAb multimer removal, the column is typically equilibrated with neutral buffer containing a high concentration of chaotropic salts (Ammonium or sodium sulfate being the most common). The load is also adjusted to have a similar concentration of chaotropic salts and under these conditions the monomer and multimers can bind to the HIC resin. The product is typically eluted from the column using a linear gradient or step to a buffer containing lower concentrations of the chaotropic salt (on no salt at all). In general, the multimer is more strongly bound to the column and elutes at a lower salt concentration, either as a separate resolved peak or as a shoulder on the tailing side of the monomer peak. HIC can also be operated in flowthrough mode under conditions where the multimers bind strongly to the column while monomeric product passes through the column with little or no binding. While multimer removal using HIC chromatography is common in mAb purifications, applying HIC chromatography to remove multimers in rpAb mixtures is not as straight-forward. Since each mAb has a different number of hydrophobic amino acids, or a varying surface hydrophobicity profile, it is not obvious that optimal conditions can be selected such that multimers are removed while individual mAbs are not selectively removed from the rpAb mixture.

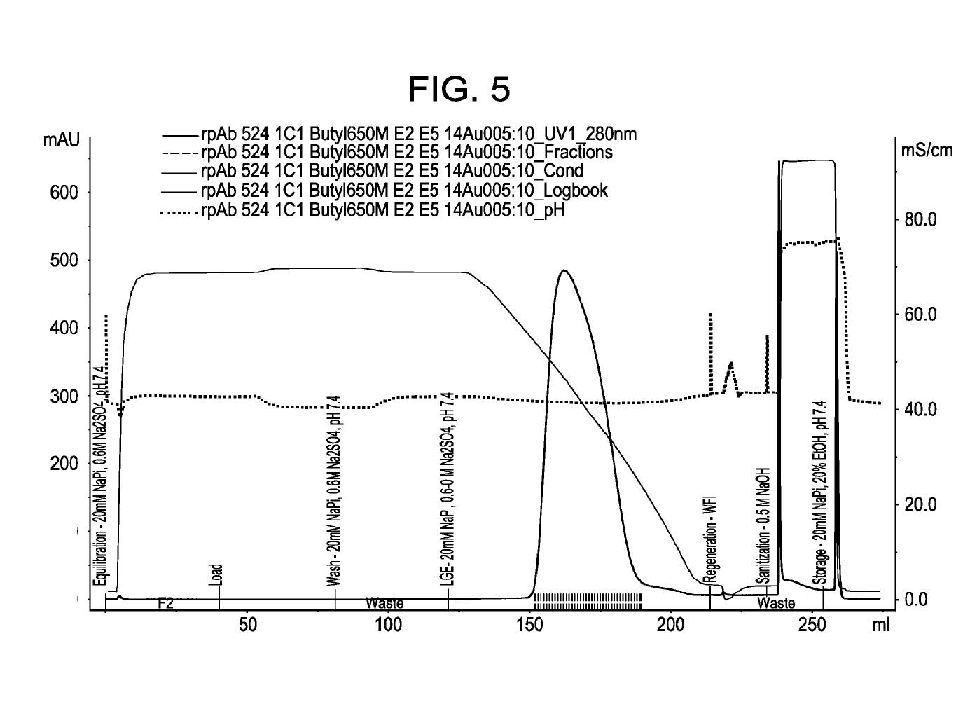

To test the ability of HIC chromatography to remove multimers in rpAb mixtures, we investigated purification of a mixture of mAb A and B (in an approximate ratio of 1:1) using Toyopearl Butyl 650M resin. The column was operated in bind and elute mode with a linear gradient of decreasing sodium sulfate concentration from 0.6 M to 0 M sodium sulfate. In this example, the multimers in the mixture are from both mAbs, in similar levels (i.e. ˜3.2% multimers from each mAb). The combined multimer level measured in the load was 6.3%. FIG. 5 shows the Butyl chromatogram of the rpAb mixture. As can be seen in the chromatogram, the individual mAbs co-elute in a single peak with no separation of the mAbs observed. If there was separation of the individual mAbs, multiple peaks with approximately similar areas would have been observed (as seen in the CEX profile in FIG. 1). A small peak eluting on the tailing side of the monomer peak was observed, and this peak was shown to be multimers by SEC-HPLC. This example had a monomer yield of 93.2%. Table 6 summarizes the load and pool analytical data. As can be seen in Table 6, multimers were reduced from 6.3% in the load, to 0.3% in the HIC pool. At the same time, the ratio of mAb B to mAb A (B:A) remained relatively constant before (0.98) and after (1.00) Butyl 650M chromatography. Thus, HIC is capable of separating multimers from monomer without simultaneously separating the individual mAbs.

TABLE 6
Summary of Butyl chromatography
of rpAb mixtures of mAb A and B.

| | SEC-HPLC | mAb ratio | Monomer Yield |
|---|---|---|---|
| Sample | (% Multimer) | (D:C) | (%) |
| Load | 6.3% | 0.98 | — |
| Pool | 0.3% | 1.00 | 93.2% |

Control of multimeric species during mAb purification is important due to the known immunogenicity of multimeric species. It is anticipated that control of multimeric species will be required in production of rpAbs for human use. Unlike mAbs, it is expected that rpAb therapeutics will have an additional constraint that the ratio of individual mAbs must be controlled in a narrow range. Thus, multimers and multimers must be removed while maintaining the ratio of individual component mAbs.

For mAb production, multimer levels are routinely controlled with ion exchange chromatography; however, multimer control in rpAb mixtures using CEX will not be feasible in many cases due to the charge heterogeneity among the individual mAbs. Other chromatographic techniques such as hydrophobic interaction, apatite, and multi-modal chromatography have been previously employed for mAb multimer removal, however, as these modalities tend to be more selective than ion-exchange, it was anticipated that these techniques would separate the component monomers of an rpAb mixture when attempting to separate multimeric species. Quite unexpectedly, we discovered that the opposite results are observed. Experiments demonstrated that hydrophobic interaction, apatite, and multimodal chromatography could retain individual mAb ratios in an rpAb mixture within a narrow range while separating undesirable multimers.

In this work we have demonstrated the ability of multi-modal, apatite, and hydrophobic interaction chromatography to be used for rpAb multimer removal. Using two or three mAb mixtures, we showed the ability of each mode of chromatography to remove greater than 2.5% multimers (in some cases multimers from multiple mAb species) to produce an rpAb product that was >99% monomer. At the same time we were able to maintain desired mAbs ratios (before and after chromatography) within 10%.

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A method of separating recombinant polyclonal antibody multimers with minimal separation of monomers comprising subjecting a mixture comprising a plurality of monoclonal antibodies to at least one separation process selected from the group consisting of multi-modal chromatography, apatite chromatography, and hydrophobic interaction chromatography thereby producing an antibody monomer preparation that is substantially free of multimers.

2. The method of claim 1 wherein the mixture is subjected to at least two separation processes selected from the group consisting of multi-modal chromatography, apatite chromatography, and hydrophobic interaction chromatography thereby producing an antibody monomer preparation that is substantially free of multimers.

3. The method of claim 1 wherein the separation process is multi-modal chromatography.

4. The method of claim 1 wherein the separation process is apatite chromatography.

5. The method of claim 1 wherein the separation process is hydrophobic interaction chromatography.

6. The method of claim 2 wherein the separation process is multi-modal chromatography and apatite chromatography.

7. The method of claim 2 wherein the separation process is multi-modal chromatography and hydrophobic interaction chromatography.

8. The method of claim 2 wherein the separation process is apatite chromatography and hydrophobic interaction chromatography.

9. The method of claim 1 wherein the mixture is subjected to multi-modal chromatography, apatite chromatography, and hydrophobic interaction chromatography thereby separating recombinant polyclonal antibody multimers with minimal separation of monomers.

10. The method of any of the preceding claims wherein said antibody preparation is at least 90% to 91% free of multimers.

11. The method of any of the preceding claims wherein said antibody preparation is at least 92% to 93% free of multimers.

12. The method of any of the preceding claims wherein said antibody preparation is at least 94% to 95% free of multimers.

13. The method of any of the preceding claims wherein said antibody preparation is at least 96% to 97% free of multimers.

14. The method of any of the preceding claims wherein said antibody preparation is at least 98% to 99% free of multimers.

15. The method of any of the preceding claims wherein said antibody preparation is 100% free of multimers.

16. The method of any of the preceding claims wherein the amount of any antibody monomer relative to any other antibody monomer in the rpAb mixture changes by less than 40%.

17. The method of any of the preceding claims wherein the amount of any antibody monomer relative to any other antibody monomer in the rpAb mixture changes by less than 30%.

18. The method of any of the preceding claims wherein the amount of any antibody monomer relative to any other antibody monomer in the rpAb mixture changes by less than 20%.

19. The method of any of the preceding claims wherein the amount of any antibody monomer relative to any other antibody monomer in the rpAb mixture changes by less than 10%.

20. The method of any of the preceding claims wherein the amount of any antibody monomer relative to any other antibody monomer in the rpAb mixture changes by less than 5%.

21. The method of any of the preceding claims wherein the amount of any antibody monomer relative to any other antibody monomer in the rpAb mixture changes by 0%.

22. A method of separating recombinant polyclonal antibody multimers with minimal separation of monomers comprising contacting a mixture comprising a plurality of monoclonal antibodies to a multi-modal chromatography resin and eluting antibody monomers from said resin with at least one elution buffer comprising a buffer species and a salt between 0 and 1 M.

23. The method of claim 22 wherein said multi-modal chromatography resin comprises a ligand with both hydrophobic and ion exchange moieties.

24. The method of claim 23 wherein said multimodal chromatography resin is a Capto Adhere chromatography resin.

25. The method any of claims 22 to 24 wherein said monomers are eluted in a linear or step-wise gradient of salt

26. The method of any of claims 22 to 24 wherein the monomers are eluted from the column with a single concentration of salt.

27. A method of separating recombinant polyclonal antibody multimers without separation of monomers comprising contacting a mixture comprising a plurality of monoclonal antibodies to an apatite chromatography resin and eluting antibody monomers from said resin with a stepwise change or linear gradient in a salt to increase conductivity from less than 1 mS/cm to greater than 90 mS/cm or any range in-between 1 mS/cm and 90 mS/cm.

28. The method of claim 27 wherein said apatite chromatography is hydroxyapatite chromatography.

29. The method of claim 27 or 28 wherein said salt is sodium chloride.

30. A method of separating recombinant polyclonal antibody multimers with minimal separation of monomers comprising contacting a mixture comprising a plurality of monoclonal antibodies to a hydrophobic interaction chromatography resin and eluting antibody monomers from said resin with a stepwise change or linear gradient in a salt to decrease conductivity from greater than 200 mS/cm to less than 1 mS/cm or any range in-between 200 mS/cm and 1 mS/cm.

31. The method of claim 30 wherein said salt is sodium sulfate.
